(19) United States
(12) Patent Application Publication
(10) Pub. No.: US 2008/0275387 A1
(43) Pub. Date: Nov. 6, 2008

(54) HEMOSTATIC MEDICAL DEVICE

(76) Inventors: Stephen C. Yeadon, Hampton, NJ (US); Robert W. Van Holten, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/743,943

(22) Filed: May 3, 2007

(51) Int. Cl. A61M 37/00

(52) U.S. Cl. 604/82

(57) ABSTRACT

A hemostatic medical device comprising a tubular syringe body extending along an axis and having a front end and a rear end; a plunger axially slidable in the body; a stem projecting axially rearward out of the body from the plunger; a free piston slidable in the body forward of the plunger and subdividing the body forward of the plunger into a front compartment at the front body end and a rear compartment between the plunger and the piston; the syringe body being formed with a mechanism to allow the contents in the rear and front compartments to mix; sterile saline in the rear compartment; and a hemostatic substance in the front compartment.

### FIELD OF THE INVENTION

The present invention relates to a hemostatic medical device that is pre-filled with a hemostatic substance and a fluid, which is set up for mixing immediately before use.

### BACKGROUND OF THE INVENTION

Hemostatic substances in solid, sponge, cake, foam or powder form, are commercially available and are used in surgical procedures. Such hemostatic substances when mixed with fluid, can be prepared in various forms depending on the contemplated end-use. For example, where higher concentrations of fluid are employed, a paste or slurry that is useful as a flowable, extrudable and injectable hemostat may be prepared for use in diffuse bleeding, particularly from uneven surfaces or hard to reach areas. Such pastes or slurries are typically prepared at the point of use by manual agitation and mixing of the hemostatic substance and a fluid to provide a uniform hemostat. The uniform hemostat is then placed into a delivery means or applicator, e.g. a syringe, and applied to the wound. Although the hemostatic substance is sterilized prior to preparing such pastes or slurries, manual agitation or mixing of the hemostatic substance with the fluid may compromise the sterility of the hemostatic substance. Therefore there is a need to minimize the manual handling of the hemostatic substance by providing a hemostatic medical device that is pre-filled with the hemostatic substance and fluid, which is available to the surgeon at the point of use.

### SUMMARY OF THE INVENTION

Described herein is a hemostatic medical device comprising a tubular syringe body extending along an axis and having a front end and a rear end; a plunger axially slidable in the body; a stem projecting axially rearward out of the body from the plunger; a free piston slidable in the body forward of the plunger and subdividing the body forward of the plunger into a front compartment at the front body end and a rear compartment between the plunger and the piston; the syringe body having a means for allowing the contents in the rear and front compartments to mix; sterile fluid in the rear compartment; and a hemostatic substance in the front compartment.

### BRIEF DESCRIPTION OF THE DRAWING

The FIGURE depicts the hemostatic medical device.

### DETAILED DESCRIPTION

A standard syringe has a tubular body with a front end adapted to receive a needle or cannula and a rear end formed with radially projecting finger braces. A plunger is axially displaceable in the body and has a rod projecting axially rearwardly from the rear end so that, when the plunger is advanced, liquid is expressed through the needle mounted on the front end.

In a first embodiment, the hemostatic medical device is a prefilled dual chamber syringe having a piston that is provided in the body forward of the plunger and subdivides the body in a starting position into a front compartment that is filled with a hemostatic substance and a rear compartment that is filled with a fluid. Immediately forward of the piston in the starting position is a means for allowing the contents in the rear and front compartments to mix, such as a bypass formed as an inwardly open and axially extending groove, a membrane optionally having one or more O-rings or support where the membrane may be ruptured, pierced or displaced, or a combination thereof. Thus when the plunger is advanced, the fluid in the rear compartment moves the piston forward until the rear end of the bypass groove is exposed in the rear compartment. Further advance of the plunger forces the fluid in the rear compartment past the piston into the front compartment where it mixes with the hemostatic substance therein. When the plunger comes to rest on the rear face of the piston and all of the fluid in the rear compartment has been driven through the bypass into the front compartment, further advance of the plunger pushes the piston forward and expresses the hemostat from the front end of the syringe body.

The hemostatic substance takes some time to be reconstituted, so screw threads may be provided between at least a portion of the plunger rod and the syringe body. Thus during at least the initial stages of advance of the plunger, the rod must be screwed into the syringe body ensuring slow and deliberate advance that gives the hemostatic substance time to mix.

Optionally, structure at the rear body end forms a radially inwardly open angularly limited cutout and at least two axially spaced, angularly offset, and radially outwardly projecting stop bumps on the stem are axially displaceable through the cutout in respective angularly offset positions of the stem. The stop bumps are axially engageable against the structure except when the stem is in the respective angular position. An elastically deformable brake element engaged between the body and the stem for axially slowing axial forward advance of the stem.

Thus with this system the brake element will prevent the plunger from advancing too rapidly, so that the fluid in the rear compartment will be pumped at a slow uniform rate around the piston through the bypass to the front compartment. The stop bumps stop advance of the piston and plunger, requiring the user to twist the piston stem to align those stop bumps striking the body rear end with the cutouts for further advance. Thus once the syringe has been fitted with a needle, cleared of air, inserted into a wound cavity, the user can simply push down on the stem until the next set of stop bumps arrests its further advance, automatically dispensing a metered dose of the hemostat. A further dose can be administered once the stem is angularly indexed, and so on until the syringe is empty. The rearmost set of stop bumps includes at least one extra bump so that they define a frontmost end position for the piston and plunger.

The stop bumps are provided in pairs with the bumps of each pair diametrically opposite each other but axially level with each other. The structure is formed with two such cutouts diametrically opposite each other. Normally each pair is offset by 90° to the preceding and following pairs.

They are spaced apart by a distance that is exactly that necessary to express a predetermined dose from the syringe. In fact the prefilled syringes can be provided with stems having differently spaced bumps, each such stem having an identifying color so that a user will know what the standard dose for a given syringe is according to its stem color. The doses can therefore be administered without looking.

The stop bumps include a frontmost stop bump that is in axial engagement with the structure when the plunger is axially forwardly engaged with the piston. Thus the user will know exactly when the rear compartment has been emptied and will not further advance the stem and waste the often valuable hemostat.

The stop bumps are axially uniformly spaced along the stem and have generally radially extending end flanks. The brake element is a forwardly directed flexible lip. The cutouts can be rectangular, seen axially, or formed as sectors.

The bumps can also have angled front flanks and rear flanks extending in planes generally perpendicular to the axis, like sawteeth. The stem is formed with an axially extending row of bumps engageable with the brake element. These bumps also are of sawtooth shape with an angled front flank and a perpendicular rear flank and the element is a flexible lip extending radially inward and axially forward from the body rear end.

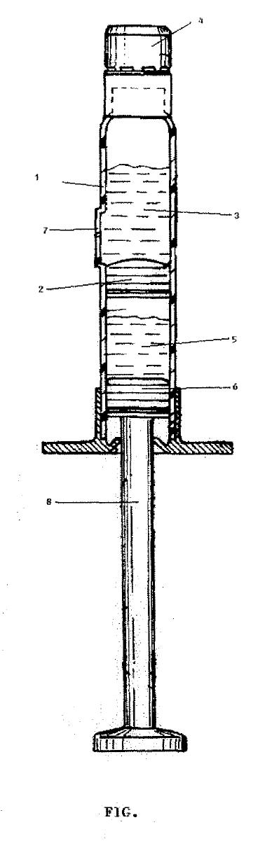

As shown in FIG. 1, the hemostatic medical device is a prefilled syringe having a substantially tubular glass or plastic body 1 centered on a center axis and slidably receiving a plunger 8. A separating means 2 (e.g. piston, stopper, membrane) subdivides the tube 1 forward of the plunger 8 into a front compartment 3 that is filled with a hemostatic substance in cake or powder form, and a rear compartment 5 that is filled with a fluid. A radially inwardly open and axially extending bypass groove 7 is formed in the tube 1 forward of the separating means 2. Initially the front end of the tube 1 is covered by a cap 4, which may be replaced for use by a needle or cannula or a syringe connector.

In a second embodiment, a hemostatic medical system comprises a first standard syringe having a tubular body prefilled with the hemostatic substance, having a front end adapted to receive another syringe, a needle or a cannula and a rear end formed with radially projecting finger braces. A plunger is axially displaceable in the body and has a rod projecting axially rearwardly from the rear end so that, when the plunger is advanced, the content thereof is expressed through the front end. The system further comprises a second syringe, prefilled with fluid such as sterile saline or flowable gelatin that may be connected to the first syringe. The plunger on the second syringe may be advanced to express the fluid into the tubular body of the first syringe, thereby reconstituting the hemostatic substance in the first syringe. The plungers on the first and second syringes may be alternately advanced to facilitate further mixing of the hemostatic substance. Upon complete mixing, the hemostat is expressed into either of the first or second syringe; the empty syringe is detached from the hemostat-containing syringe; and a needle or a cannula is attached to the hemostat-containing syringe. The hemostat may then be expressed directly to or into the wound site.

In a third embodiment, a hemostatic medical system comprises the prefilled dual chamber syringe described in the first embodiment above, which has a front end adapted to receive another syringe, a needle or a cannula. The system further comprises a second syringe, prefilled with fluid such as flowable gelatin, that may be connected to the first syringe, after the fluid in the rear compartment has been expelled into the front compartment and the hemostatic substance therein has been reconstituted. The plunger on the second syringe may be advanced to express the fluid into the tubular body of the first syringe, thereby forming a mixture of the hemostat and gelatin. The plungers on the first and second syringes may be alternately advanced to facilitate further mixing of the hemostat and gelatin. Upon complete mixing, the hemostat/gelatin mixture is expressed into either of the first or second syringe; the empty syringe is detached from the hemostat/gelatin mixture containing syringe; and a needle or a cannula is attached to the hemostat/gelatin mixture containing syringe. The hemostat/gelatin mixture may then be expressed directly to or into the wound site.

Hemostatic substances that may be used in the prefilled hemostatic medical device include, without limitation, procoagulant enzymes, proteins and peptides, can be naturally occurring, recombinant, or synthetic, and may be selected from the group consisting of prothrombin, thrombin, recombinant human thrombin (rh thrombin), fibrinogen, fibrin, fibronectin, heparinase, Factor X/Xa, Factor VII/VIIa, Factor IX/IXa, Factor XI/XIa, Factor XII/XIIa, tissue factor, serine protease, kallikrein, batroxobin, ancrod, ecarin, von Willebrand Factor, collagen, elastin, albumin, gelatin, platelet surface glycoproteins, selectin, procoagulant venom, plasma, plasminogen activator inhibitor, platelet activating agents, synthetic peptides having hemostatic activity, derivatives of the above and any combination thereof. Preferred hemostatic substances used in the present invention are thrombin, fibrinogen and fibrin.

Lyophilization or spray freeze drying of hemostatic substance is essential in order to obtain required shelf life without refrigeration being required upon storage. Excipients such as glycine, mannitol sucrose, and trehalose may incorporated with the hemostatic substance to protect the protein during freezing and thawing and their rapid reconstitution rate when exposed to water, buffer or other reconstitution solution.

For example, the front compartment of the dual chamber syringe may be filled with the solution of the hemostatic substance and saline. After the fill of the front compartment, the hemostatic solution may be frozen by loading on a pre-chilled shelf or more preferably by freezing the product by immersion in dry ice. The hemostatic solution is preferably frozen at least 10 to 20 C. below the phase transition temperature of the solution.

Annealing is usually performed on an as needed basis. Based on the nature of the hemostastic substance, annealing or thermal cycling prior to primary drying may be used as a way to increase the primary drying rate and reduce the effect of non-homogeneity in nucleation rates. Primary drying is initiated by reducing the pressure in the lyophilized chamber. Pressure is reduced below the saturated vapor pressure of ice at the frozen product temperature, leading to sublimation. The sublimation step can take place in a number of different conditions and is custom designed for the protein formulation being dried increasing the shelf temperature increases the energy available to drive sublimation.

Shelf temperature is then raised to ambient temperature and held until the target moisture is reached which is ˜5% as measured by Karl Fischer assay. At the correct moisture content of the cake in the syringe barrels the first chamber is capped to stop moisture influx or cake disruption on breaking the vacuum.

The lyophilization process includes but is not limited to formulation, freezing, annealing, primary drying, and secondary drying. These lyophilization steps will vary dependent on the formulation, the container material and the configuration of the product container. With a dual chamber syringe the formulated hemostatic solution is resting on a stopper instead of the bottom of a glass vial resulting in a temperature lag between the product and the shelf. Target shelf temperatures, chamber pressures and time parameters can be significantly affected by this temperature lag. The constraint of the syringe diameter also affects the robustness of the process. By varying the volume of the hemostatic solution, the height of the liquid changes resulting in the process being modified, resulting in a change in the final product characteristics. In order to have varying dose in a dual chamber syringe it may require that the protein activity in the formulation is increased or decrease so that the fill volume change is not required in varying the dose put-up.

The actual cycle used in lyophilization of the first chamber of the dual chamber syringe is dependent on the lyophilizer equipment, the fill volume, and the formulation of the material along with the number of components in the chamber. An example of a lyophilization cycle would be as follows:

| | Pressure | Time | Target Temp |
|---|---|---|---|
| Lyophilization step | (ubars) | (hours) | (° C.) |
| Chamber load | 1,013,000 | <1 | RT |
| Freezing | 1,013,000 | <2 | −50 |
| Sublimation | 133 | >20 | −25 |
| level 1 | | | |
| Sublimation | 133 | >10 | −15 |
| level 2 | | | |
| Sublimation | 133 | >15 | 20 |
| Level 3 | | | |
| Secondary drying | 10 | >15 | 25 |
| Nitrogen flash ramp | 1,013,000 | 1 | 25 |
| Capping | 1,013,000 | <1 | RT |

Alternatively, the hemostatic substance may be processed by spray freeze drying, which combines atomization to create droplets, freezing as a result of contacting a freezing medium for example liquid nitrogen and removal of moisture by sublimation under vacuum. This technique produces uniform sized powders of proteins without a secondary procedure to pulverize the lyophilization cake. These particles will have specific moisture content and mean diameter dependent on the conditions, which they were formed under. This powder can be filled into the primary chamber by a swizzle stick technique where a tube of a defined inner diameter is placed into the powder reservoir and dispenses a predetermined weight.

Filling of the front compartment of the dual chamber syringe takes place after the preparation of syringe components. The syringe, barrels, rubber stoppers and closure parts are all washed and sterilized prior to use. Filling is performed using various pump technologies, rotary piston pump, peristaltic pump etc. The syringe barrel is placed upside down and the first stopper is inserted. The first hemostatic solution in this case thrombin solution formulated for lyophilization is sterile filtered and filled to a specified volume. The syringe is then lyophilized resulting in a protein cake. The syringe is then crimped to seal the first chamber.

Fluids that may be prefilled in the rear compartment of the hemostatic medical device include but are not limited to sterile saline, water for injection, deionized water, enzyme solutions or other compatible fluids

Fluids that may be prefilled in the second syringe of the hemostatic medical system of the second and third embodiments include but are not limited to sterile saline, water for injection, deionized water, enzyme solutions, other compatible fluids and or flowable hemostats include but are not limited to gelatins, proteins or polysaccharides.

We claim:

1. A hemostatic medical device comprising:
(a) a tubular syringe body extending along an axis and having a front end and a rear end;
(b) a plunger axially slidable in the body;
(c) a stem projecting axially rearward out of the body from the plunger;
(d) a free piston slidable in the body forward of the plunger and subdividing the body forward of the plunger into a front compartment at the front body end and a rear compartment between the plunger and the piston,
(e) the syringe body having a means for allowing the contents in the rear and front compartments to mix;
(f) sterile fluid in the rear compartment; and
(g) a hemostatic substance in the front compartment.

2. The hemostatic medical device of claim 1, wherein the means for allowing the contents in the rear and front compartments to mix is a bypass passage in the syringe body.

3. The hemostatic medical device of claim 1, further comprising structure at the rear body end forming a radially inwardly open angularly limited cutout; at least two axially spaced, angularly offset, and radially outwardly projecting stop bumps on the stem axially displaceable through the cutout in respective angularly offset positions of the stem, the stop bumps being axially engageable against the structure except when the stem is in the respective angular position; and means including an elastically deformable brake element engaged between the body and the stem for axially slowing axial forward advance of the stem.

4. The hemostatic medical device of claim 3, wherein the stop bumps are provided in pairs with the bumps of each pair diametrically opposite each other but axially level with each other, the structure being formed with two such cutouts diametrically opposite each other.

5. The hemostatic medical device of claim 3, wherein the stop bumps are offset by about 90° to each other relative to the axis.

6. The hemostatic medical device of claim 3, wherein the stop bumps include a frontmost stop bump that is in axial engagement with the structure when the plunger is axially forwardly engaged with the piston.

7. The hemostatic medical device of claim 3, wherein the stop bumps are axially uniformly spaced along the stem.

8. The hemostatic medical device of claim 3, wherein the brake element is a forwardly directed flexible lip.

9. The hemostatic medical device of claim 3, wherein the bumps have angled front flanks and rear flanks extending in planes generally perpendicular to the axis.

10. The hemostatic medical device of claim 3, wherein the stem is formed with an axially extending row of bumps engageable with the brake element.

11. The hemostatic medical device of claim 10, wherein the bumps are of sawtooth shape with an angled front flank and a perpendicular rear flank and the element is a flexible lip extending radially inward and axially forward from the body rear end.

12. The hemostatic medical device of claim 3, wherein the cutout has generally radially extending end flanks.
